(19) United States
(12) Patent Application Publication
(10) Pub. No.: US 2024/0382540 A1
(43) Pub. Date: Nov. 21, 2024

(54) MODULATION OF FUSOBACTERIUM VARIUM TO PREVENT BOVINE LIVER ABSCESSES AND IMPROVE CATTLE PERFORMANCE

(71) Applicant: Jacques Mathieu, Houston, TX (US)

(72) Inventors: Jacques Mathieu, Houston, TX (US); Pedro Alvarez, Houston, TX (US); Cory Schwarz, Houston, TX (US)

(73) Assignee: Sentinel Environmental Group, LLC, Houston, TX (US)

(21) Appl. No.: 18/317,422

(22) Filed: May 15, 2023

(51) Int. Cl. A61K 35/76; A61K 39/00; A61K 39/114; A61P 31/04

(52) U.S. Cl. CPC A61K 35/76; A61K 39/114; A61P 31/04; A61K 2039/552

(57) ABSTRACT

The present invention relates to methods and compositions for treating cattle to modulate the abundance of Fusobacterium varium in the gastrointestinal tract with the intent to reduce liver abscess occurrence or severity, or to improve cattle growth or feed efficiency.

### PRIOR RELATED APPLICATIONS

This application claims the benefit of U.S. Provisional Application No. 63/364,738, filed on May 16, 2022, the content of which is incorporated herein by reference in its entirety.

### FEDERALLY SPONSORED RESEARCH STATEMENT

This invention was made with government support under contract no. 2025980 awarded by the National Science Foundation (NSF) and project no. 1820015 awarded by the United States Department of Agriculture (USDA). The government has certain rights in the invention.

### FIELD OF THE DISCLOSURE

The present invention relates generally to the field of veterinary medicine, and more particularly to methods and compositions for mitigating bovine liver abscesses by targeting Fusobacterium varium in the gastrointestinal tract of cattle using various approaches including antibacterial agents, bacteriophages, vaccines, dietary modifications, probiotic supplementation, antimicrobial peptides or proteins, and genetic manipulation of Fusobacterium varium.

### BACKGROUND OF THE DISCLOSURE

The beef cattle industry is under increasing regulatory and public pressure to reduce or eliminate the use of antibiotic feed additives as they are considered to be a major factor driving the widespread proliferation of antibiotic-resistant bacteria. Globally, antibiotic resistance was associated with almost 5 million deaths and was the direct cause of 1.3 million deaths in 2019. However, in-feed antibiotics provide significant economic and animal welfare benefits, including the prevention of liver abscesses in feedlot cattle. Even with antibiotic use, it has been estimated that liver abscesses currently cost the beef cattle industry over $60 million per year; a number which would be expected to substantially increase if in-feed antibiotics were withdrawn. Additionally, revenue losses or increased costs can have resonating effects in the beef cattle industry, which already suffers from low profit margins and high market volatility. This dichotomy between public welfare and the economic sustainability of the beef cattle industry has created a growing need for feedlot management to identify and implement alternatives to in-feed antibiotics.

Fusobacterium necrophorum is a Gram (−), aerotolerant anaerobic bacteria that is commonly isolated from abdominal abscesses and respiratory tract infections in animals, and is widely considered the primary etiologic agent of liver abscesses in cattle. It is considered a normal inhabitant of bovine rumen, and contains two major subspecies: Fusobacterium necrophorum subsp. fundiliforme and Fusobacterium necrophorum subsp. necrophorum. Subspecies necrophorum strains are generally considered to be more virulent for cattle and have been shown to produce greater amounts of leukotoxin. In cattle liver abscesses, Fusobacterium necrophorum may act synergistically with Trueperella pyogenes, Salmonella enterica, and other bacteria, which are considered secondary etiologic agents. While Fusobacterium necrophorum is almost always present in liver abscesses, they are not always the most abundant phyla present (Pinnell et al., 2022, Front. Microbiol). Nevertheless, Fusobacterium necrophorum has been the exclusive focus of interventions developed to prevent bovine liver abscesses and reduce their severity.

Ruminal concentrations of Fusobacterium necrophorum are elevated in grain-fed cattle but are significantly reduced by the antibiotics tylosin and virginiamycin when provided as a component of the cattle feed. The use of these antibiotics in cattle feed is reported to reduce the occurrence of liver abscesses by approximately 40-70% in feedlot cattle. However, the use of in-feed antibiotics may negatively affect the growth of beneficial and commensal bacterial species while promoting the growth of bacteria harboring antibiotic resistance genes. Thus, more selective and targeted alternatives to antibiotics are desirable to maintain healthy rumen and gut microbiomes and to mitigate the unintentional proliferation of antibiotic resistant bacteria due to antibiotic overuse.

Various alternatives to the currently used antibiotics have been developed and tested for the purpose of reducing ruminal concentrations of Fusobacterium necrophorum and preventing liver abscesses. These include, but are not limited to vaccines, essential oils, direct-fed microbials, fermentation byproducts, antimicrobial peptides and proteins, and bacteriophages. Other methods for selectively inhibiting bacterial species could also be applied, such as CRISPR-based genome editing, though not as a standalone technology. However, to date, no alternative to in-feed antibiotics for liver abscess prevention has been widely adopted by the cattle industry, with a lack of efficacy being the most commonly cited reason.

The present invention is based on our discovery that Fusobacterium varium is both highly prevalent and abundant in cattle rumen and may contribute the formation and severity of bovine liver abscesses (Schwarz et al., 2023, J. Anim. Sci.). Prior to our work described herein, there has never been a report or scientific publication suggesting any other Fusobacterium species besides Fusobacterium necrophorum plays a role in bovine liver abscess formation.

Importantly, we also discovered that Fusobacterium varium strains isolated from the bovine rumen are highly resistant to tylosin, which is the most widely used intervention to prevent bovine liver abscesses. They are also resistant to the commonly used ionophore monensin, have broad and variable metabolic capacities that overlap with Fusobacterium necrophorum, and harbor a wide array of virulence factors. We also observed substantial overlap between the metabololic capacities of Fusobacterium necrophorum and Fusobacterium varium, though their genomes are highly divergent based on average nucleotide identities (ANI). This is significant because it suggests that Fusobacterium varium may directly compete with Fusobacterium necrophorum for resources and has the potential to influence Fusobacterium necrophorum abundance or virulence, but is not controlled by currently used antibiotic feed additives. These new findings open up the possibility for novel prevention methods targeting Fusobacterium varium, potentially providing a more effective approach to reduce the incidence of bovine liver abscesses and improve cattle health and productivity.

### SUMMARY OF THE DISCLOSURE

The present invention pertains to novel methods for preventing and treating bovine liver abscesses by specifically targeting Fusobacterium varium (F. varium). The invention is based on the surprising and unexpected discovery that F. varium, a bacterium previously overlooked in studies of bovine rumen microbiome, is a common and significant member of the bovine rumen bacterial community, displaying high resistance to conventionally used antibiotics, and may play a crucial role in liver abscess formation in cattle.

The invention includes methods that leverage this new understanding of F. varium's role and characteristics. These methods may involve the use of specific antibiotics, bacteriophages, or other treatments that are effective against F. varium. In particular, the invention encompasses the use of bacteriophages that specifically target F. varium, or antibiotics to which F. varium is susceptible, offering a unique and effective strategy for controlling its population in the bovine rumen.

The invention also includes methods that consider the metabolic capabilities of F. varium, such as its ability to utilize lysine, maltose, and lactate. Dietary adjustments or supplements that affect the availability of these metabolites in the rumen may be utilized as part of these methods. Moreover, modulating F. varium abundance could provide a method for improving cattle feed efficiency.

This invention provides a significant shift in the approach to the prevention and treatment of bovine liver abscesses, presenting a non-obvious and inventive step over existing practices which have exclusively focused on F. necrophorum. This new approach has the potential to significantly improve bovine health and productivity by addressing a previously overlooked factor contributing to liver abscesses and rumen metabolism.

### BRIEF DESCRIPTION OF THE DRAWINGS

FIG. 1 shows the species composition of the Fusobacterium genus in cattle rumen fluid.

FIG. 2 shows the reduction in Fusobacterium varium following treatment with the phage φKent.

FIG. 3 shows the inhibition of Fusobacterium varium by uncharacterized bacterial strains isolated from cattle rumen fluid.

### DETAILED DESCRIPTION

The present invention provides a method for preventing or treating bovine liver abscesses by specifically targeting Fusobacterium varium (F. varium), a bacterium previously overlooked in studies of the bovine rumen microbiome, but found to be a significant player in the bovine rumen ecosystem and potentially in the formation of liver abscesses.

The traditional focus of liver abscess prevention methods in cattle has been on Fusobacterium necrophorum (F. necrophorum). However, the present invention is based on a series of unexpected and non-obvious discoveries that challenge the prevailing understanding and point to F. varium as a contributing factor in bovine liver abscess formation and severity.

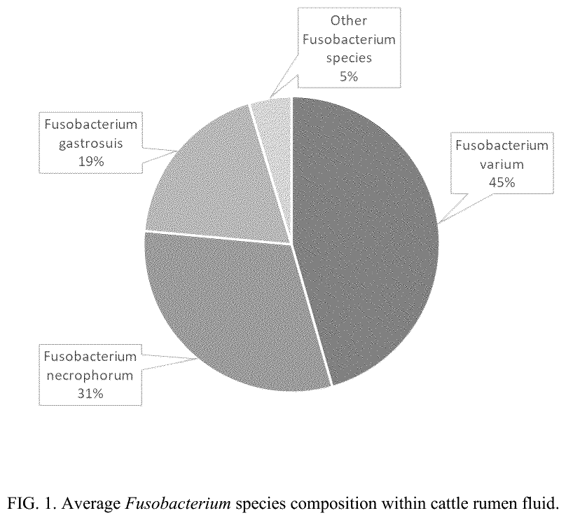

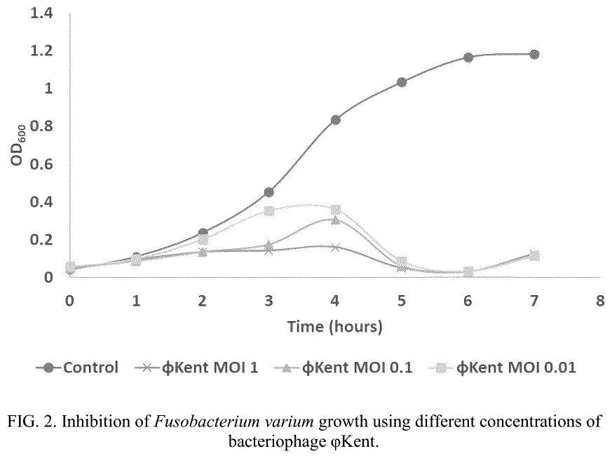

The inventors have found that F. varium is a common and widespread member of the bovine rumen bacterial community, contrary to the long-held consensus belief within the cattle industry and animal science community that F. necrophorum is the dominant member of the genus, and the only member of the genus to be associated with liver abscesses (FIG. 1). This discovery was made through culture methods using selective agar and 16S rRNA metagenomic analysis, identifying F. varium in every sample evaluated, regardless of the cattle's diet, antibiotic exposure, or liver abscess status. These results were confirmed by the isolation and characterization of numerous F. varium strains directly from over 100 rumen fluid samples. Notably, F. varium was also detected directly in abscessed liver tissue. Additionally, we have isolated bacteriophages that are lytic against F. varium from the majority of rumen fluid samples tested (FIG. 2).

It is likely that the presence of F. varium was previously overlooked due to deficiencies in the methodologies used. For example, several studies used a most-probable-number (MPN) method to determine F. necrophorum concentrations in rumen fluid. This method relied on a selective media in combination with a colorimetric indole assay to determine the presence of F. necrophorum in serially-diluted samples. At the time, it was not known that F. varium was a ubiquitous ruminal native, capable of growing in the same selective media as well as providing a positive result in indole tests. Moreover, targeted metagenomics studies historically relied on short-read sequencing that were incapable of distinguishing species, so all Fusobacterium were assumed to be F. necrophorum.

The inventors further discovered that F. varium displays a high level of resistance to antibiotics commonly used in feedlots, including tylosin, the current gold standard for liver abscess reduction strategies in cattle, and monensin, which is used to improve feed efficiency. This antibiotic resistance stands in stark contrast to F. necrophorum, which is generally susceptible to these antibiotics. This finding suggests that currently used antibiotic feed additives are insufficient in effectively managing F. varium.

Preliminary genomic analysis of F. varium isolates from the rumen revealed the presence of virulence genes related to those of pathogenic human F. varium isolates associated with active invasion of mammalian cells. One isolate, F. varium KL10, contained almost twice as many genes classified as virulence factors than F. necrophorum ATCC 25286. Such genes included adhesion factors (e.g., Fap2) required for host colonization, two-component secretion systems, antibiotic resistance genes, and genes associated with active host cell invasion. This finding further suggests a potentially significant role of F. varium in liver abscess development.

These unexpected discoveries constitute a new paradigm in the understanding of bovine rumen microbiology and liver abscess formation. The inventors have determined that F. varium is not just an incidental inhabitant of the bovine rumen, but a resident bacterium that may play a significant role in bovine health, particularly in the development of liver abscesses.

Currently it is not known if F. varium has been erroneously overlooked in North American cattle populations, or if it was only recently acquired. Notably, F. varium has been associated with ulcerative colitis in Japanese patients but is not frequently reported in North America or Europe. In support of the recent emergence of F. varium as a pathogen in Asia, a rapid increase in the proportion of Fusobacterium infections in Korean patients caused by F. varium since only 2016 was reported (Lee et al., 2022, PLOS One). Additionally, F. varium was shown to cause more severe infections than F. nucleatum and F. necrophorum, demonstrated by high rates of in-hospital mortality (12.5%) and ICU admission (26.8%) as well as acute kidney injury (14.3%).

Microbial lactate utilization is linked to several pathogenic mechanisms. It has been repeatedly stated in prior research that F. varium is incapable of utilizing lactate, though it has been shown to produce lactate, which can be utilized by F. necrophorum. However, we have demonstrated that the presence of lactate in rumen fluid enrichment cultures strongly selects for F. varium, enriching native strains up to 35,000-fold. Currently it is unknown if some or all cattle-associated F. varium strains are able to derive energy from lactate. However, our analysis of F. varium genomes revealed the presence of a composite transposon in the human isolate Fv113-g1 containing genes related to lactate utilization that are also linked to microbial pathogenicity and host resistance. Thus, it is possible that the acquisition of this or a similar mobile genetic element has provided some bovine F. varium strains with the capability of utilizing lactate as a source of energy and a means for avoiding host immunity.

Regardless of whether F. varium is a net producer or consumer of lactate, the presence of this species in cattle rumen is likely to impact the abundance and behavior of F. necrophorum and consequently play a role in liver abscess formation. Thus, modulating the abundance of F. varium could be used as a strategy to indirectly prevent liver abscesses through disruption of interactions with F. necrophorum.

The Fusobacterium genus has also been shown to be negatively correlated to feed efficiency in cattle. Both F. varium and F. necrophorum are known to metabolize various amino acids, including lysine, which is considered a limiting nutrient for cattle. Some isolated strains have been described as hyper-ammonia-producing bacteria due to the high rates at which they generate ammonia from amino acids, and these bacteria are considered detrimental to cattle feed efficiency. As F. varium is more abundant, more frequently present, resistant to tylosin and monensin, and faster growing than F. necrophorum, it is likely that it has more significant impact on feed efficiency, though this has not been previously proposed due to the widespread oversight of this species presence in cattle rumen.

Various approaches can be used to selectively alter the abundance of F. varium in cattle rumen, and can be broadly classified as top-down or bottom-up approaches. Bottom-up strategies generally involve development of synthetic communities using pure cultures that are pre-designed to achieve a specific function. While such approaches have seen some niche success in industrial fermentations or waste processing, contamination is a major challenge and our understanding of how to best engineer such communities is still limited. Thus, such approaches are not practical for cattle rumen microbiome engineering. In contrast, top-down approaches rely on modifying a pre-existing microbial community and have seen extensive practice in the cattle industry, where diet and various xenobiotics (e.g., antibiotics, prebiotics, essential oils) are used to enhance animal performance through their effects on the rumen microbiome. Other methods that impact the rumen microbiome in a top-down manner include probiotics (direct-fed microbials), rumen transfaunation or microbiota transplants, bacteriophages, antimicrobial peptides and proteins, and vaccines.

Given the new understanding of F. varium abundance and prevalence in the bovine rumen, as well as its role in rumen ecology, the inventors have developed methods for preventing and treating bovine liver abscesses and enhancing cattle feed efficiency that take into account the unique properties of F. varium. These methods may involve the use of specific antibiotics, bacteriophages, or other treatments that are effective against F. varium. In particular, these methods may include the use of bacteriophages that specifically target F. varium, or antibiotics to which F. varium is susceptible, offering an effective strategy for controlling its population in the bovine rumen. The prevention and treatment methods may also consider the metabolic capabilities of F. varium, such as its ability to utilize lysine, maltose, and lactate. For example, dietary adjustments or supplements that affect the availability of these metabolites in the rumen could be implemented. These newly developed methods represent a non-obvious and inventive step over existing practices which have focused primarily on F. necrophorum, ignoring the presence and potential importance of F. varium.

In the preferred implementation, bacteriophage cocktails are administered to cattle with feed or water. Doses may be administered at any interval or time, but it is recommended to begin administration simultaneous with the transition from a forage-based diet to concentrated feed. For example, a bacteriophage cocktail consisting of a lyophilized powder may be added to daily cattle rations using a micro dosing machine, which is typically used in feedlots to add micro-ingredients such as antibiotics. Bacteriophage cocktails may also be added to water or provided directly by oral dosing. In order to facilitate storage and application, bacteriophage cocktails may be stabilized by encapsulation, lyophilization, or any other method that protects the bacteriophages from degradation and maintains viability.

In another implementation, bacterial strains capable of either promoting or suppressing the growth of F. varium are administered to cattle with feed or as direct-fed microbials (e.g., oral gavage). In general, bacterial strains that display greater selectivity in interactions towards F. varium are preferred. Ruminal isolates are preferred to increase the probability of survival within cattle gastrointestinal tracts.

In another implementation, said bacteriophages and bacterial strains are administered to cattle in combination to increase the impact on Fusobacterium varium abundance. Bacteriophages may also be applied that are able to infect native rumen bacterial species that compete with or have the potential to prevent colonization or engraftment of the administered bacterial strains. In this manner, bacteriophages may help clear ecological niches to enhance probiotic persistence with the objective of modulating F. varium abundance.

### Example 1: Isolation and Identification of Fusobacterium varium from Bovine Rumen Fluid

In the course of our efforts to isolate ruminal strains of F. necrophorum using both direct plating of rumen fluid and plating following enrichment in lactate broth onto Fusobacterium-selective medium (Brazier et al., 1991), we surprisingly identified most isolates as F. varium. This identification was based on 16S rRNA sequencing using close to the full-length gene (27F/1492R primers), a reliable method for determining bacterial species (FIG. 1).

We collected 165 ruminal fluid samples from processing plants for the purpose of bacteriophage isolation, and nearly half of these were also used for bacterial isolation. Astonishingly, F. varium was identified in every single sample evaluated. Further screening of 15 rumen fluid samples from a USDA facility revealed the presence of F. varium, irrespective of the tylosin feeding regimen.

When we directly streaked ruminal fluid onto Fusobacterium-selective medium (FAA-JVN), approximately 50% of the strains were identified as Fusobacterium. Further species-level identification via 16S rRNA sequencing revealed that between 50-75% of these isolates were F. varium. The remaining isolates were generally either F. necrophorum or F. gastrosuis. Interestingly, plating lactate enrichments onto the same medium most often resulted in the identification of F. varium, particularly when 8-10 colonies were randomly selected for colony PCR (cPCR).

In order to fully understand the community composition and structure, as well as the relative abundances of different Fusobacterium species in the ruminal fluid and various enrichment cultures, we employed targeted 16S metagenomics. To achieve species-level resolution, we generated 16S rRNA amplicons using a close to full-length, barcoded primer set (27F/1492R) and sequenced them using the Oxford Nanopore MinION system.

The community analysis confirmed the presence of Fusobacterium in unenriched ruminal samples, with the genus-level characterization being consistent with previous studies (Henderson et al., 2015). Specifically, Fusobacterium showed a relative abundance between 0.001% and 0.003% in ruminal fluid. However, such low abundance (1 Fusobacterium read per 50,000 total reads) posed a challenge to accurately assess species distribution.

To overcome this, we also generated amplicons from rumen samples using genus-specific primers. This method significantly increased sequencing depth within the genus, allowing us to better assess the distribution of Fusobacterium species within each sample. Of the 98,575 classified reads from 12 ruminal fluid samples, an astounding 98,052 were assigned to Fusobacterium, demonstrating the specificity of this approach. With this method, F. varium emerged as the most abundant member of the genus, comprising on average 46% of Fusobacterium species and 0.00138% of the total microbial community (FIG. 1).

The second most abundant species was F. necrophorum (31% and 0.00093%), followed by F. gastrosuis (19% and 0.00057%). The remaining 5% (or 0.00015% of the total community) comprised a collection of other Fusobacterium species, including F. ulcerans, F. nucleatum, F. equinum, F. simiae, F. mortiferum, F. gonidiaformans, F. periodonticum, F. russi, and F. canefelium, listed in order of decreasing abundance.

These findings significantly expand our understanding of the ruminal microbiome and particularly the prevalence and distribution of Fusobacterium species. Despite the previously assumed dominance of F. necrophorum in this environment, our data clearly indicate that F. varium is a highly prevalent and historically underappreciated member of the bovein rumen microbial community.

In conclusion, our work reveals the ubiquitous presence of F. varium in the rumen fluid samples analyzed, highlighting its potential importance within the rumen microbiome. This could have implications for the management of bovine health and the prevention of conditions like acidosis and liver abscesses, which are associated with Fusobacterium infections. Further research is needed to investigate the specific roles of F. varium and other Fusobacterium species in these conditions, and how they might be effectively targeted for treatment or prevention.

### Example 2: Antimicrobial Susceptibility Testing of Fusobacterium Species

In an effort to understand the differential response of Fusobacterium species to antimicrobial agents commonly used in livestock, we examined the effect of tylosin and monensin on the growth of reference strains and newly isolated strains of F. varium and F. necrophorum.

To set up the experiment, we exposed three strains each of F. varium and F. necrophorum to tylosin and monensin at concentrations relevant to ruminal environments. Our observations revealed striking differences between the two species in their responses to the tested antimicrobials.

For F. varium, none of the tested strains exhibited substantial growth inhibition in the presence of tylosin. The most notable reduction was observed in strain F. varium DNLA-8, which displayed a 13% decline in growth. Similarly, F. varium strains did not show substantial growth inhibition when exposed to monensin. Only a 15% decrease in growth was observed in the bovine isolates, and the reference strain F. varium NCTC 10560 showed no significant response to monensin.

In contrast, the F. necrophorum strains displayed a more pronounced susceptibility to both tylosin and monensin. All three F. necrophorum strains tested exhibited over 67% growth inhibition in response to tylosin, with strains 8L1 and FNA showing the most considerable declines of 92% and 94%, respectively (P<0.05). When exposed to monensin, growth reductions of more than 87% were observed for all F. necrophorum strains. Strains A and 8L1 were most affected, with both displaying a 98% reduction in growth. Interestingly, the growth of F. necrophorum was slightly less inhibited by monensin when grown in a medium containing only casein pancreatic digest and yeast extract as carbon sources. In this context, growth reductions of 44-66% were observed (P<0.05).

Overall, our data demonstrate that F. varium strains are largely resistant to tylosin and monensin, while F. necrophorum strains are generally more susceptible, with their response varying based on strain and growth medium. These findings suggest that the current practices used to control F. necrophorum, such as the administration of tylosin and monensin, may not be effective against F. varium. This has significant implications for the management of bovine health and could inform the development of more effective strategies for controlling Fusobacterium infections.

### Example 3: Genomic Insights into the Ecological Role and Virulence Potential of Fusobacterium varium Isolates in the Bovine Rumen

To better understand the ecological role and potential virulence of F. varium in the bovine rumen, we performed whole-genome sequencing on two isolates of this bacterium: F. varium 1701-2, derived from a grain-fed cow, and F. varium KL10, from a feedlot steer. F. varium 1701-2 exhibited strong growth on maltose compared to other strains of the same species, while F. varium KL10 thrived well in a PY medium without lactate.

The genomes of the two isolates were found to be consistent in size with previously reported human F. varium isolates, with KL10 and 1701-2 being approximately 3.36 Mb and 3.44 Mb, respectively. The GC content of these isolates, 29.3% for KL10 and 29.5% for 1701-2, was also in line with those of other F. varium strains. Furthermore, the average nucleotide identities (ANI) between our strains and the F. varium NCTC 10650 reference strain exceeded 98%, affirming their classification within the same species.

Initial genome analysis of our isolates using the RAST annotation pipeline identified virulence-associated subsystems in both strains, as well as in previously sequenced human isolates. Interestingly, a slightly higher proportion of genes related to virulence, disease, and defense were identified in our bovine isolates compared to those in F. necrophorum ATCC 25286 and F. varium NCTC 10560. However, the large proportion of unassigned open reading frames suggests a need for a more detailed investigation of potential virulence-related genes.

Both of our F. varium isolates contained sequences similar to two-partner secretion systems (TPS), known to play key roles in virulence. These TPS had the highest similarity to virulence factor genes within the broader Fusobacterium genus. Further investigation using various databases identified similarities to other virulence genes such as antibiotic resistance genes, autotransporters, and “active invader” associated proteins. Additionally, both strains contained genes similar to the Mycobacterium virulence operon potentially involved in quinolinate biosynthesis.

In terms of metabolic capacity, both isolates harbored D- and L-lactate dehydrogenases, located near FMN-dependent ABC transporters, suggesting they can metabolize lactate. However, the absence of lactate permeases, present in other Fusobacterium genomes, suggests differences in lactate uptake mechanisms. Similarly, while both isolates contained aminomutases involved in lysine degradation, they lacked the lysine permease found in other Fusobacterium strains, indicating potential variations in lysine metabolism.

In conclusion, our study sheds light on the genomic characteristics of F. varium strains in the bovine rumen, pointing to their potential role in this complex ecosystem. Furthermore, the presence of multiple virulence-associated genes and subsystems signifies the potential pathogenic capabilities of these strains. However, comprehensive functional analyses of these genes and their products are required to fully understand the pathogenic potential and ecological roles of these bacteria in the rumen.

### Example 4: Isolation and Characterization of Ruminal Bacteria with Antagonistic Activity Towards Fusobacterium varium

In this study, we aimed to identify and isolate ruminal bacteria that exhibit antagonistic activity towards F. varium. Individual strains of F. varium were cultured and used to prepare double agar overlays. We serially diluted rumen fluid and aliquoted select dilutions onto these overlays. After incubation at 37° C., the overlays were examined daily for the emergence of new colonies, specifically those showing zones of inhibition indicative of suppression or lysis of F. varium.

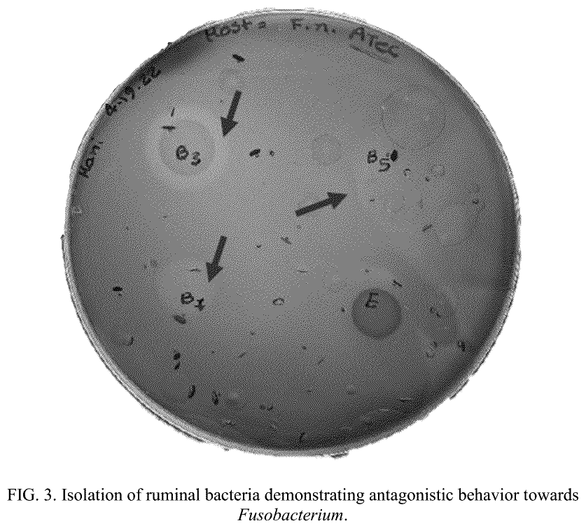

Colonies demonstrating this inhibitory activity were picked and restreaked on new agar plates to ensure purity. These purified colonies were once again streaked onto double agar overlays containing F. varium to confirm their antagonistic activity (FIG. 3). The inhibitory colonies were then isolated, and their identities were determined via 16S rRNA gene sequencing.

We further assessed the inhibitory spectrum of each isolate against a panel of cattle rumen strains, which included F. varium, F. necrophorum, Streptococcus bovis, Enterococcus faecalis, Enterococcus faecium, Escherichia coli, and Selenomonas ruminantium. Interestingly, most of our isolated strains demonstrated inhibitory activity towards more than one strain of F. varium, but had limited or no effect on the other bacterial species tested. However, one isolate stood out as it demonstrated broad-spectrum activity, inhibiting several strains of both F. varium, F. necrophorum, and S. bovis. Many of these antagonistic strains were identified as belonging to the Bacillus genus.

These results provide a promising starting point for the development of microbial interventions in the rumen. The identified antagonistic bacteria could potentially be used as probiotics to control the growth of detrimental Fusobacterium species, thereby improving ruminal health and productivity in cattle. Further studies are required to explore the mechanism of this antagonistic activity and to evaluate the potential of these isolates in in vivo conditions.

### Example 5: Isolation and Characterization of Fusobacterium varium Bacteriophages from Bovine Rumen Fluid

Initially, bacteriophage pools were prepared from rumen fluid samples and filtered lysates from enrichment cultures containing lactate or lysine. In some cases, a combination of antibiotics including josamycin, vancomycin, and norfloxacin was added to enhance the selection for Fusobacterium. These bacteriophage pools were then processed using a fraction-based approach, involving clarification of the supernatant via centrifugation and subsequent filtration through a 0.45 μm PVDF filter. The filtrate was divided into two fractions: one third was further filtered through a 0.22 μm filter to yield enveloped and nonenveloped phages below 220 nm, while another third was treated with chloroform to lyse residual bacteria and yield nonenveloped phages below 450 nm. Both these fractions were used in spot assays to verify the presence of phages. The remaining fraction, containing both enveloped and nonenveloped phages below 450 nm, was reserved for plaque assays.

Bacteriophages capable of lysing F. varium strains in the rumen were isolated via spot or plaque assays and subsequently purified. Their host ranges were confirmed via spot tests on F. varium strains isolated from various sites and cattle rumen fluid samples. By using lactate or lysine enrichment prior to bacteriophage pool preparation, we successfully isolated F. varium bacteriophages from most of the rumen fluid samples, given they were appropriately preserved. Preservation of samples was done by mixing rumen fluid samples in a 1:1 ratio with phosphate-buffered saline containing 14% DMSO, achieving a final DMSO concentration of 7%.

In certain samples, oxyrase was also added to remove residual oxygen. Samples were quickly processed and stored at −80° C. for optimal preservation. If not preserved promptly, the isolation of F. varium bacteriophages occasionally failed.

This method confirmed the ubiquity of F. varium bacteriophages in cattle rumen, indicating they can be a convenient source for ongoing isolation efforts. Having a readily available source of bacteriophages is crucial as bacteriophage cocktails are typically used for microbial control to ensure a wide host coverage and minimize bacterial resistance development. The compatibility of these phages was assessed using Appelmans' method by challenging host strain cultures with bacteriophage pairs.

Bacteriophage activity against F. varium strains was evaluated in a 96-well plate format using absorbance at 600 nm. Most of the bacteriophages showed lytic activity against multiple strains and exhibited strong inhibitory activity for at least 7 hours. Genome sequencing of these bacteriophages revealed both temperate and lytic bacteriophages. Moreover, the majority of the F. varium bacteriophages yielded high titers of at least 10⁹ PFU/mL in culture lysates, which is significant for reducing production costs and ensuring a satisfactory return on investment for cattle producers using feed additives.

### REFERENCES

Pinnell, Lee J., et al. “Not all liver abscesses are created equal: the impact of tylosin and antibiotic alternatives on bovine liver abscess microbial communities and a first look at bacteroidetes-dominated communities.” Frontiers in Microbiology 13 (2022).
Schwarz, Cory, et al. “Unexpected finding of Fusobacterium varium as the dominant Fusobacterium species in cattle rumen: Potential implications for liver abscess etiology and interventions.” Journal of Animal Science (2023): skad130.

What is claimed is:

1. A method for mitigating bovine liver abscesses, the method comprising targeting Fusobacterium varium in the gastrointestinal tract of cattle by administering an effective amount of an antibacterial agent.

2. The method of claim 1, wherein the antibacterial agent is specifically effective against Fusobacterium varium that demonstrated resistance to conventional in-feed antibiotics.

3. The method of claim 1, wherein the bacteriophage is specifically lytic against Fusobacterium varium.

4. The method of claim 1, wherein the antibacterial agent or bacteriophage is delivered through the cattle's feed or drinking water.

5. The method of claim 1, wherein the antibacterial agent or bacteriophage is encapsulated, lyophilized, or spray dried prior to delivery.

6. The method of claim 1, wherein the targeting further comprises administering a vaccine specifically designed against Fusobacterium varium.

7. The method of claim 6, wherein the vaccine is administered through injection or oral administration.

8. The method of claim 1, wherein the targeting further comprises implementing a dietary modification, probiotic supplementation, or administering antimicrobial peptides or proteins.

9. The method of claim 1, wherein the targeting further comprises genetic manipulation of Fusobacterium varium to reduce its virulence or liver abscess formation.

10. The method of any one of claims 1 to 9, further comprising evaluating the effectiveness of the Fusobacterium varium-targeted interventions by comparing the incidence of liver abscesses in treated and untreated cattle.

11. A composition for mitigating bovine liver abscesses, comprising an antibacterial agent or bacteriophage specifically effective against Fusobacterium varium, wherein the composition is formulated for delivery via feed or drinking water in a lyophilized, spray dried, or encapsulated form.

12. The composition of claim 11, further comprising a vaccine specifically targeting Fusobacterium varium.

13. A method for improving cattle health and productivity, and reducing the incidence of liver abscesses, comprising the steps of claim 1.

14. The method of claim 13, applied specifically in the dairy cattle industry.

15. The method of claim 13, applied specifically in the beef cattle industry.
